(19) United States
(12) Patent Application Publication
(10) Pub. No.: US 2001/0000173 A1
(43) Pub. Date: Apr. 5, 2001

(54) Antioxidants for the stabilization of formulations comprising surfactants

(76) Inventors: Dietmar Huglin, Eimeldingen (DE); Erich Kramer, Basel (CH)

Correspondence Address: CIBA SPECIALTY CHEMICALS CORPORATION, PATENT DEPARTMENT, 540 WHITE PLAINS RD, P O BOX 2005, TARRYTOWN, NY, 10591-9005, US

(21) Appl. No.: 09/734,234

(22) Filed: Dec. 7, 2000

Related U.S. Application Data
(63) Continuation of application No. 09/298,571, filed on Apr. 23, 1999, now abandoned.

(30) Foreign Application Priority Data
Apr. 28, 1998 (EP) 98810374.3
Oct. 23, 1998 (CH) 2143/98

(51) Int. Cl. A61K 7/40

(52) U.S. Cl. 510/130; 510/131

(57) ABSTRACT

The invention relates to cleaning compositions comprising

(a₁) a phenolic antioxidant of the formula (1) and/or (2); and/or

(a₂) an antioxidant of the formula (3); and

(b) a surfactant comprising a long alkyl or alkenyl chain.

The antioxidants used according to the invention have excellent reactivity, good stability to hydrolysis, particularly in an alkaline medium, and, because of their solubility, can be easily incorporated into the soap formulations.

1. Solid and liquid soaps have been used for cleaning human skin for a long time. The stability of the soap composition is an important criterion for problem-free use or a long shelf life.

2. It is known that free radical reactions adversely affect the stability of a soap composition. Free radicals initiate chain reactions which effect the decomposition of the long-chain hydrocarbon chains of the soaps, free acids or synthetic surfactants and the like in cleaning compositions. Such reactions can also bring about other negative effects, such as, for example, discoloration and rancidification.

3. Degradation of the long hydrocarbon chains can be prevented in cleaning compositions by adding antioxidants, such as, for example, butylated hydroxytoluene (BHT), which either prevent the catalysis of certain free radical mechanisms or, as free radicals, terminate the free radical chain reaction.

4. However, the use of BHT causes stability problems in the soap compositions, such as, for example, decoloration, or yellow-brown by-products form.

5. WO 97/27839 discloses soap compositions in which the stabilizers used are specific phenolic antioxidants. However, these compounds have poor solubility and can only be incorporated with difficulty.

6. The object of the invention was thus to find phenolic antioxidants which have better solubility in soap formulations and can be incorporated without problem into the corresponding soap compositions.

7. The present invention thus provides a cleaning composition comprising

8. (a₁) a phenolic antioxidant of the formula

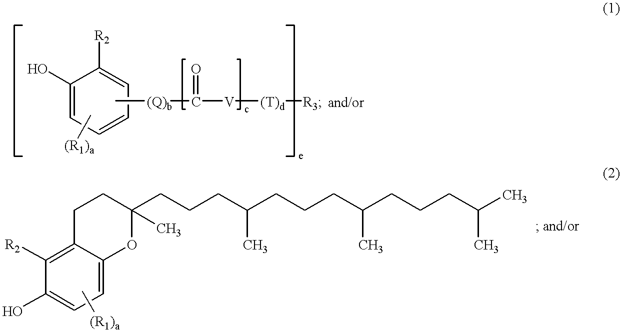

9. (a₂) an antioxidant of the formula

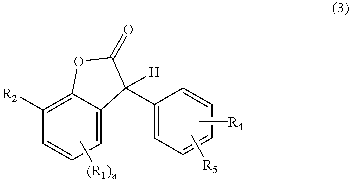

10. in which in the formulae (1), (2) and (3)

11. R₁ is hydrogen; C₁-C₂₂ alkyl; C₁-C₂₂ alkylthio; C₅-C₁₂ cycloalkyl; phenyl; or C₇-C₉ phenylalkyl;

12. R₂ is C₁-C₂₂ alkyl; C₅-C₁₂ cycloalkyl; phenyl; C₇-C₉ phenylalkyl; or —SO₃M;

13. Q is —CₘH₂ₘ—;

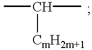

14. —CₘH₂ₘ—NH; a radical of the formula

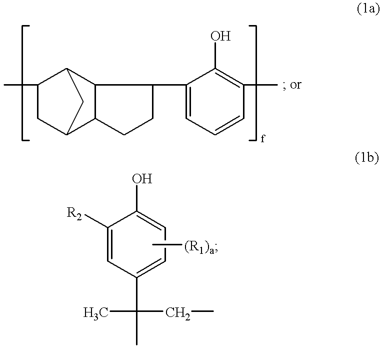

15. T is —CₙH₂ₙ—; —(CH₂)ₙ-O-CH₂—;

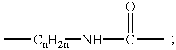

16. or a radical of the formula

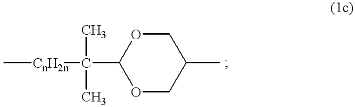

17. V is —O—; or —NH—;

18. a is 0; 1; or 2;

19. b, c and d independently of one another are 0; or 1;

20. e and f independently of one another are an integer from 1 to 3; and

21. m, n and p independently of one another are an integer from 1 to 3;

22. when e is 1,

23. R₃ is hydrogen; M; C₁-C₂₂ alkyl; C₅-C₁₂ cycloalkyl; C₁-C₂₂ alkylthio; C₂-C₂₂ alkenyl; C₁-C₁₈ phenylalkyl; a radical of the formula

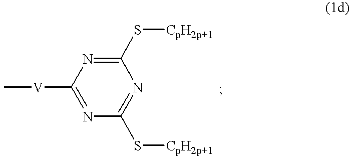

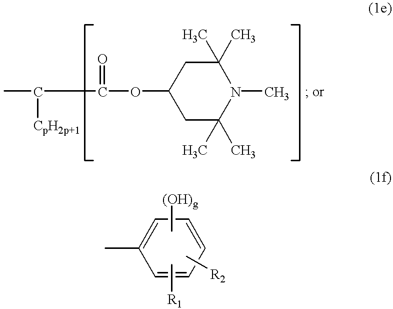

24. where, when R₃ is C₁-C₂₂ alkyl, b=0; or Q is a radical of the formula (1a) or (1b);

25. g is 0 or 1;

26. M is alkali metal; ammonium;

27. when e is 2,

28. R₃ is a direct bond; —CH₂—;

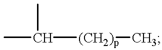

29. —O—; or —S—;

30. when e is 3,

31. R₃ is a radical of the formula

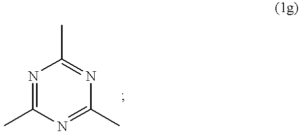

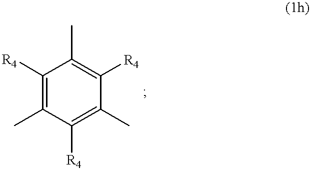

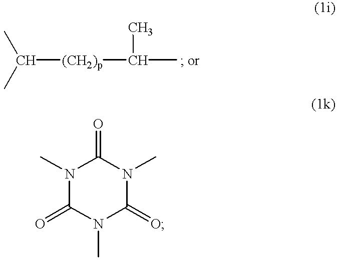

32. where, when R₃ is a radical of the formula (1k), c=1; and;

33. R₄ and R₅ independently of one another are hydrogen; or C₁-C₂₂ alkyl; and

34. (b) a surfactant comprising a long alkyl or alkenyl chain.

35. C₁-C₂₂-Alkyl are straight-chain or branched alkyl radicals, such as, for example, methyl, ethyl, n-propyl, isopropyl, n-butyl, sec-butyl, tert-butyl, amyl, isoamyl or tert-amyl, heptyl, octyl, isooctyl, nonyl, decyl, undecyl, dodecyl, tetradecyl, pentadecyl, hexadecyl, heptadecyl, octadecyl or eicosyl.

36. C₁-C₂₂-Alkylthio are straight-chain or branched alkylthio radicals, such as, for example, methylthio, ethylthio, n-propylthio, isopropylthio, n-butylthio, sec-butylthio, tert-butylthio, amylthio, heptylthio, octylthio, isooctylthio, nonylthio, decylthio, undecylthio, dodecylthio, tetradecylthio, pentadecylthio, hexadecylthio, heptadecylthio, octadecylthio or eicosylthio.

37. C₂-C₁₈-Alkenyl is, for example, allyl, methallyl, isopropenyl, 2-butenyl, 3-butenyl, isobutenyl, n-penta-2,4-dienyl, 3-methyl-but-2-enyl, n-oct-2-enyl, n-dodec-2-enyl, isododecenyl, n-dodec-2-enyl or n-octadec-4-enyl.

38. C₅-C₇-Cycloalkyl is cyclopentyl, cycloheptyl or, in particular, cyclohexyl.

39. C₇-C₉-Phenylalkyl is phenylpropyl, phenylethyl and, in particular, benzyl.

40. In the novel composition, preference is given to using antioxidants of the formula (1) in which

41. Q is —CₘH₂ₘ—, and in particular a methylene or ethylene radical and

42. m is as defined in formula (1).

43. In particular, V in formula (1) is —O—.

44. Of particular interest in the novel composition are compounds of the formula (1) in which R₁ and R₂ independently of one another are C₁-C₂₂ alkyl, and in particular C₁-C₅ alkyl.

45. Furthermore, there is also particular interest in compounds of the formula (1) in which

46. a is 1.

47. Of very particular interest are compounds of the formula

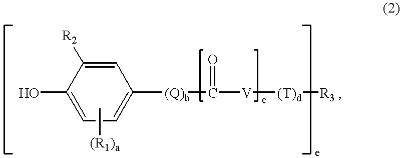

48. in which

49. R₁ and R₂ independently of one another are C₁-C₅ alkyl,

50. a is 1 or 2; and

51. R₃, Q, V, T, b, c, d and e are as defined in formula (1).

52. Particular preference is given to compounds of the formula (2) in which

53. e is 1.

54. Compounds of particular interest are those of the formula

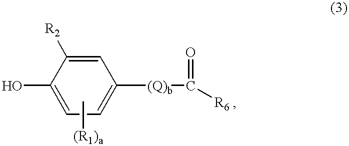

55. in which

56. R₆ is —O—M; or

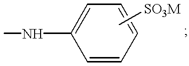

57. M is hydrogen; ammonium; or alkali metal; and

58. R₁, R₂, Q, a and b are as defined in claim 8.

59. Furthermore, component (a₁) is preferably a compound of the formula

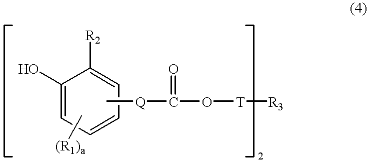

60. in which

61. R₁ and R₂ independently of one another are C₁-C₅ alkyl;

62. Q is —CₘH₂ₘ—; or —CₘH₂ₘ—NH—;

63. R₃ is a direct bond; —O—; or —S—;

64. a is 1 or 2;

65. m is 1 to 5; and

66. T is as defined in formula (1).

67. Of the compounds of the formula (3), preference is given to those in which

68. Q is ethylene;

69. R₃ is a direct bond; and

70. R₁, R₂, T and a are as defined in formula (3).

71. Very particularly preferred compounds of the formula (4) are those in which

72. T is —O—CH₂.

73. Other interesting compounds which can be used according to the invention conform to the formula

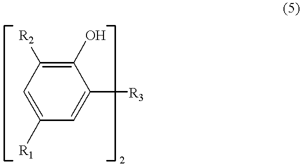

74. in which

75. R₁ and R₂ are C₁-C₅-alkyl; and

76. R₃ is —CH₂— or

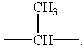

77. Of the compounds of the formula (2) to (5), preference is given to those in which

78. R₁ and R₂ are the tert-butyl radical; and

79. a is 1.

80. Furthermore, in the novel formulation, component (a₁) is preferably a compound of the formula

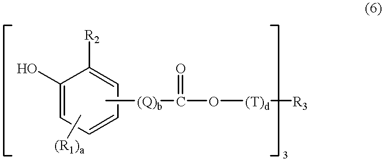

81. in which

82. Q is —CₘH₂ₘ—;

83. T is —CₙH₂ₙ—;

84. R₁ and R₂ independently of one another are C₁-C₅ alkyl;

85. R₃ is the radical of the formula (1g); (1h); (1i); or (1k);

86. m and n independently of one another are from 1 to 3;

87. a is 1 or 2; and

88. b and d independently of one another are 0 or 1;

89. and very particularly a compound of the formula

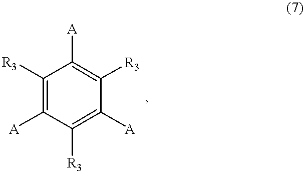

90. in which

91. A is a radical of the formula

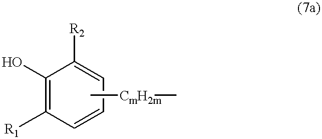

92. R₁, R₂ and R₃ independently of one another are C₁-C₅ alkyl; and

93. m is 1 to 3.

94. Component (a₁) is preferably also a compound of the formula

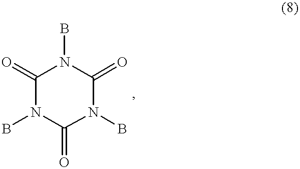

95. in which

96. B is a radical of the formula

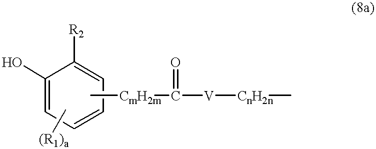

97. R₁ and R₂ independently of one another are C₁-C₅ alkyl;

98. V is —O—; or —NH—;

99. a is 1; or 2;

100. m is 1 to 3; and

101. n is 0 to 3.

102. Typical antioxidants used according to the invention are listed in Table 1:

TABLE 1

Compound of the formula
(9)
(10)
(11)
(12)
(13)
(14)
(15)
(16)
(17)
(18)
(19)
(20)
(21)
(22)
(23)
(24)
(25)
(26)
(27)
(28)
(29)
(30)
(31)
(32)
(33)
(34)

103. The antioxidants corresponding to component (a₁) and (a₂) can be used in the novel cleaning composition either as individual components or as a mixture of several individual compounds.

104. Component (a) is generally present in the novel cleaning composition in a concentration of from 50 to 1000 ppm.

105. The antioxidants used according to the invention have excellent reactivity and can thus be used advantageously at low temperatures. Furthermore, they display better stability to hydrolysis, particularly in an alkaline medium. Because of their good solubility, they can be easily incorporated into soap formulations.

106. The novel compositions thus show high stability towards colour changes and chemical decomposition. This is to be attributed to the effectiveness, colour stability, ease of incorporation and stability to hydrolysis of the antioxidants used.

107. Component (b) in the novel cleaning composition may be any surfactant which removes dirt from the skin and is at the same time sensitive to the oxidative degradation which leads to decoloration and/or unpleasant odours.

108. Suitable examples are anionic, nonionic or zwitterionic and amphoteric synthetic, detersive substances.

109. Suitable anionic detersive substances are

110. sulfates, such as, for example, fatty alcohol sulfates whose alkyl chain has from 8 to 18 carbon atoms, such as, for example, sulfated lauryl alcohol;

111. fatty alcohol ether sulfates, such as, for example, the acid esters or salts thereof of a polyadduct of from 2 to 30 mol of ethylene oxide with 1 mol of a C₈-C₂₂ fatty alcohol;

112. the alkali metal salts, ammonium salts or amine salts of C₈-C₂₀-fatty acids referred to as soaps, such as, for example, coconut fatty acid;

113. alkylamidosulfates;

114. alkylaminosulfates, such as, for example, monoethanolamine lauryl sulfate;

115. alkylamide ether sulfates;

116. alkylaryl polyether sulfates;

117. monoglyceride sulfates;

118. alkanesulfonates whose alkyl chain contains from 8 to 20 carbon atoms, for example dodecylsulfonate;

119. alkylamidosulfonates;

120. alkylarylsulfonates;

121. α-olefinsulfonates;

122. sulfosuccinic acid derivatives, such as, for example, alkylsulfosuccinates, alkyl ether sulfosuccinates or alkylsulfosuccinamide derivatives;

123. N-(alkylamidoalkyl)amino acids of the formula

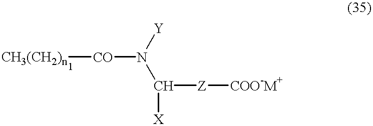

124. in which

125. X is hydrogen; C₁-C₄ alkyl or —COOM⁺;

126. Y is hydrogen or C₁-C₄ alkyl;

127. Z is —(CH₂)ₘ₁₋₁

128. m₁ is an integer from 1 to 5;

129. n₁ is an integer from 6 to 18 and

130. M is an alkali metal cation or ammonium cation;

131. alkyl and alkylaryl ether carboxylates of the formula

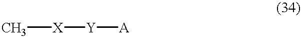

132. in which

133. X is a radical —(CH₂)₅₋₁₉—O—;

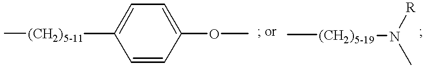

134. R is hydrogen; or C₁-C₄ alkyl;

135. Y is —(CHCHO)₁₋₅₀—;

136. A is —(CH₂)ₘ₂₋₁COO⁻M⁺; or

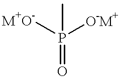

137. m₂ is from 1 to 6 and

138. M is an alkali metal cation or amine cation.

139. Other anionic surfactants used are fatty acid methyl taurides, alkylisethionates, fatty acid polypeptide condensates and fatty alcohol phosphoric esters. The alkyl radicals in these compounds preferably have from 8 to 24 carbon atoms.

140. The anionic surfactants are generally in the form of their water-soluble salts, such as the alkali metal salts, ammonium salts or amine salts. Examples of such salts are lithium, sodium, potassium, ammonium, triethylamine, ethanolamine, diethanolamine or triethanolamine salts. In particular, the sodium, potassium or ammonium (NR₁R₂R₃) salts are used, in which R₁, R₂ and R₃ independently of one another are hydrogen, C₁-C₄ alkyl or C₁-C₄ hydroxyalkyl.

141. Very particularly preferred anionic surfactants in the novel composition are monoethanolamine lauryl sulfate or the alkali metal salts of fatty alcohol sulfates, in particular sodium lauryl sulfate and the product of the reaction between from 2 to 4 mol of ethylene oxide and sodium lauryl ether sulfate.

142. Suitable zwitterionic and amphoteric surfactants are C₈-C₁₈ betaines, C₈-C₁₈ sulfobetaines, C₈-C₂₄ alkylamido-C₁-C₄ alkylenebetaines, imidazoline carboxylates, alkylamphocarboxycarboxylic acids, alkylamphocarboxylic acids (e.g. lauroamphoglycinate) and N-alkyl-b-aminopropionates or -iminodipropionates, the C₁₀-C₂₀ alkylamido-C₁-C₄ alkylenebetaines and, in particular, coconut fatty acid amidopropylbetaine being preferred.

143. Examples of suitable nonionogenic surfactants are derivatives of the adducts of propylene oxide/ethylene oxide having a molecular weight of from 1000 to 15,000, fatty alcohol ethoxylates (1-50 EO), alkylphenol polyglycol ethers (1-50 EO), ethoxylated carbohydrates, fatty acid glycol partial esters, such as, for example, diethylene glycol monostearate, fatty acid alkanolamides and dialkanolamides, fatty acid alkanolamide ethoxylates and fatty amine oxides.

144. Furthermore, component (b) may be the salts of saturated and unsaturated C₈-C₂₂ fatty acids either alone, as a mixture with one another or as a mixture with the other detersive substances mentioned as component (b). Examples of these fatty acids are capric, lauric, myristic, palmitic, stearic, arachidic, behenic, caproleic, dodecenoic, tetradecenoic, octadecenoic, oleic, eicosenoic and erucic acid, and the technical-grade mixtures of such acids, such as, for example, coconut fatty acid. These acids are in the form of salts, suitable cations being alkali metal cations, such as sodium and potassium cations, metal atoms, such as zinc and aluminium atoms, or sufficiently alkaline, nitrogen-containing organic compounds, such as amines or ethoxylated amines. These salts can also be prepared in situ.

145. Component (b) in the novel composition is preferably a soap, i.e. a branched or unbranched long-chain alkyl- or alkenyl-carboxylic acid salt, such as, for example, the sodium, potassium, ammonium or substituted ammonium salt.

146. Furthermore, in addition to components (a) and (b), the novel composition may comprise, as component (c), a light protection agent of the sterically hindered amine type.

147. This is preferably a 2,2,6,6-tetraalkylpiperidine derivative which contains at least one group of the formula

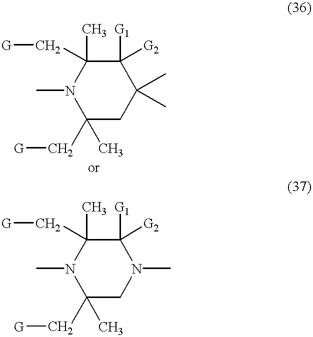

148. in which G is hydrogen or methyl, in particular hydrogen.

149. Examples of tetraalkylpiperidine derivatives which can be used as component (c) can be found in EP-A-356 677, pages 3-17, sections a) to f). Said passages of this patent are regarded as part of the present description. The following tetraalkylpiperidine derivatives are particularly advantageously used:

150. bis(2,2,6,6-tetramethylpiperidin-4-yl) sebacate, bis(2,2,6,6-tetramethylpiperidin-4-yl) succinate, bis(1,2,2,6,6-pentamethylpiperidin-4-yl) sebacate, bis(1-octyloxy-2,2,6,6-tetramethylpiperidin-4-yl) sebacate, bis(1,2,2,6,6-pentamethylpiperidyl) n-butyl 3,5-di-tert-butyl-4-hydroxybenzylmalonate, the condensate of 1-hydroxyethyl-2,2,6,6-tetramethyl-4-hydroxypiperidine and succinic acid, the condensate of N,N′-bis(2,2,6,6-tetramethyl-4-piperidyl)hexamethylenediamine and 4-tert-octylamino-2,6-dichloro-1,3,5-s-triazine, tris(2,2,6,6-tetramethyl-4-piperidyl) nitrilotriacetate, tetrakis(2,2,6,6-tetramethyl-4-piperidyl) 1,2,3,4-butanetetraoate, 1,1′-(1,2-ethanediyl)bis(3,3,5,5-tetramethylpiperazinone), 4-benzoyl-2,2,6,6-tetramethylpiperidine, 4-stearyloxy-2,2,6,6-tetramethylpiperidine, bis(1,2,2,6,6-pentamethylpiperidyl)-2-n-butyl-2-(2-hydroxy-3,5-di-tert-butylbenzyl) malonate, 3-n-octyl-7,7,9,9-tetramethyl-1,3,8-triazaspiro[4.5]decan-2,4-dione, bis(1-octyloxy-2,2,6,6-tetramethylpiperidyl) sebacate, bis(1-octyloxy-2,2,6,6-tetramethylpiperidyl) succinate, the condensate of N,N-bis(2,2,6,6-tetramethyl-4-piperidyl)-hexamethylenediamine and 4-morpholino-2,6-dichloro-1,3,5-triazine, the condensate of 2-chloro-4,6-di(4-n-butylamino-2,2,6,6-tetramethylpiperidyl)-1,3,5-triazine and 1,2-bis(3-aminopropylamino)ethane, the condensate of 2-chloro-4,6-di(4-n-butylamino-1,2,2,6,6-pentamethylpiperidyl)-1,3,5-triazine and 1,2-bis(3-aminopropylamino)ethane, 8-acetyl-3-dodecyl-7,7,9,9-tetramethyl-1,3,8-triazaspiro[4.5]decan-2,4-dione, 3-dodecyl-1-(2,2,6,6-tetramethyl-4-piperidyl)pyrrolidine-2,5-dione, 3-dodecyl-1-(1,2,2,6,6-pentamethyl-4-piperidyl)pyrrolidine-2,5-dione, a mixture of 4-hexadecyloxy- and 4-stearyloxy-2,2,6,6-tetramethylpiperidine, the condensate of N,N′-bis(2,2,6,6-tetramethyl-4-piperidyl)hexamethylenediamine and 4-cyclohexylamino-2,6-dichloro-1,3,5-triazine, the condensate of 1,2-bis(3-aminopropylamino)ethane and 2,4,6-trichloro-1,3,5-triazine and 4-butylamino-2,2,6,6-tetramethylpiperidine (CAS Reg. No. [136504-96-6]); (2,2,6,6-tetramethyl-4-piperidyl)-n-dodecylsuccinimide, (1,2,2,6,6-pentamethyl-4-piperidyl)-n-dodecylsuccinimide, 2-undecyl-7,7,9,9-tetramethyl-1-oxa-3,8-diaza-4-oxospiro[4.5]decane, the product of the reaction between 7,7,9,9-tetramethyl-2-cycloundecyl-1-oxa-3,8-diaza-4-oxospiro[4.5]decane and epichlorohydrin, tetra(2,2,6,6-tetramethylpiperidin-4-yl)butane 1,2,3,4-tetracarboxylate, tetra(1,2,2,6,6-pentamethylpiperidin-4-yl)butane 1,2,3,4-tetracarboxylate, 2,2,4,4-tetramethyl-7-oxa-3,20-diaza-21-oxodispiro[5.1.11.2]heneicosane, 8-acetyl-3-dodecyl-1,3,8-triaza-7,7,9,9-tetramethylspiro[4.5]decane-2,4-dione, or a compound of the formulae

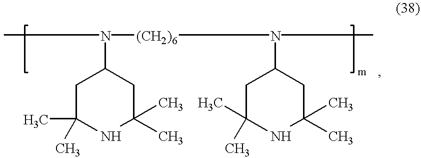

151. in which m has a value from 5-50,

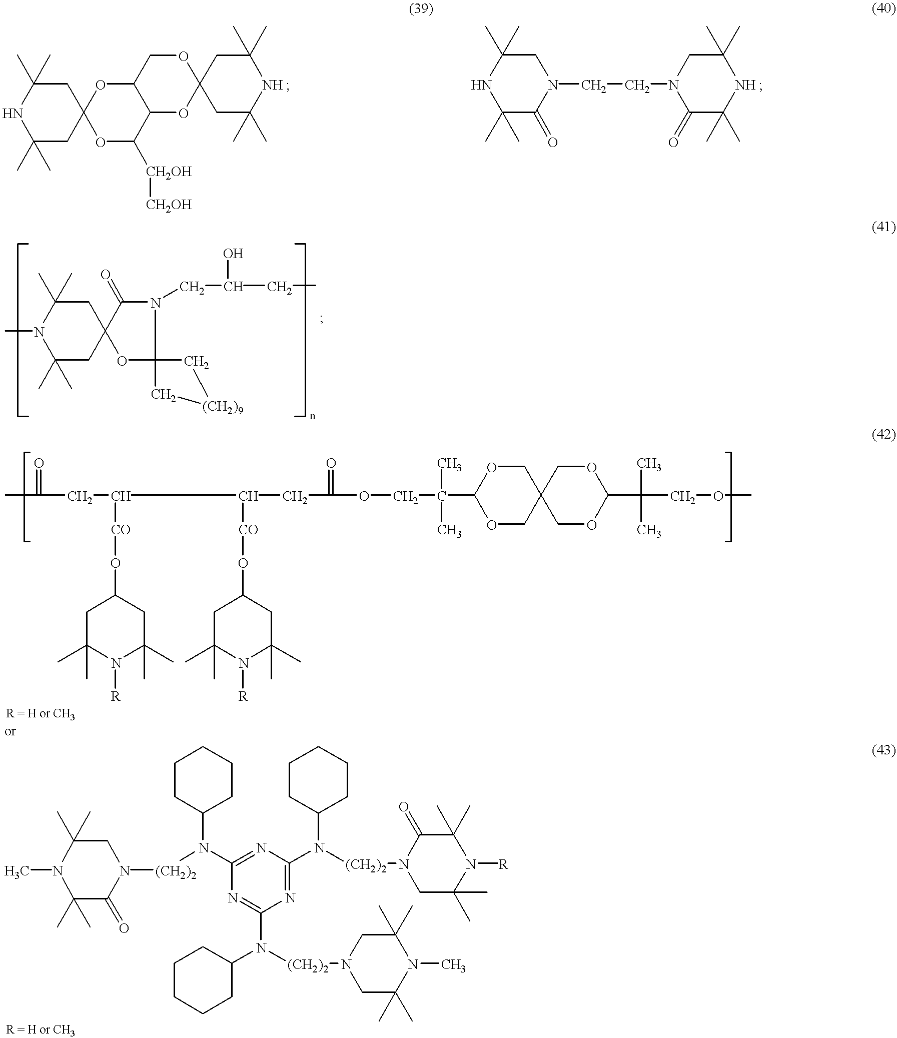

152. The novel composition can be in the form of a solid, gel, syndet or liquid soap. It can be prepared by the usual methods.

153. The soaps (solid soaps, syndets, liquid soaps) are prepared by processes which are generally customary in the soaps industry for these products and described in the literature (see, for example, L. Spitz (Ed.), Soaps and Detergents, A Theoretical and Practical Review, AOCS Press, Champaign, Ill., USA (1996)). An important factor in the preparation of solid soaps is the intensive mixing of the soap composition prior to extrusion to achieve a homogeneous distribution of the ingredients, in particular of the antioxidant. The antioxidant is usually added to the soap composition directly or, if appropriate, predissolved in perfume, homogeneously distributed therein by mixing (for example in a guide-beam mixer) and kneading (for example in an intensive kneader), before the composition is extruded or moulded. Liquid soaps are likewise produced by homogenization of the constituents in suitable mixing devices (for example Sulzer mixers, Erestat mixers or DAT mixers from Pfaudler), uniform distribution of the antioxidant generally being achieved more quickly than in the case of solid soaps as a result of the lower viscosity of the formulation. An alternative procedure involves incorporating the antioxidant into the basic soap composition (flakes, ribbons), if necessary with the application of heat (melting).

154. The following examples illustrate the invention.

155. Unless stated otherwise, parts and percentages are by weight. The temperature is given in ° C.

### PREPARATION EXAMPLES FOR SOAP COMPOSITIONS

### EXAMPLE 1

### Preparation of a Solid Soap

156.

| | Component | % by weight |
|---|---|---|
| A | Mixture of tallow fatty, coconut and palm kernel | 85 |
| | soaps (sodium salts) | |
| B | Water | ad 100 |
| C | Glycerol | 1 |
| | Titanium dioxide | 0.2 |
| | Lactic acid (88%) | 0.2 |
| | Antioxidant of the formula (14), (15), (17), (21), (22), | 0.005-0.1 |
| | (23), (25) or (28) | |
| | Disodium EDTA | 0.1 |

157. Preparation: the soap base (A) is thoroughly mixed, and the water (B) is added at 20° C. The viscous paste is homogenized using a rotor-stator, and then the components (C) are added in the order given with vigorous mixing. The mixture is homogenized for a further 15 minutes and extruded in a bench extruder. The soap bars are produced by pressing (bench press).

### EXAMPLE 2

### Preparation of a Liquid Soap

158.

| Component | % by weight |
|---|---|
| Glycerol monostearate (40%) | 5.0 |
| Ammonium lauryl sulfate (28%) | 25.0 |
| Cocoamidopropylhydroxysultaine | 3.5 |
| Disodium EDTA | 0.1 |
| Propylene glycol | 1.0′ |
| Lauryl diethanolamide | 0.5 |
| Antioxidant of the formula (14), (15), (17), (21), (22), | 0.001-0.05 |
| (23), (25) or (28) | |
| Fragrances, preservatives | Q.S. |
| Water | ad 100 |
| Citric acid | Q.S. (pH 5.5-6.5) |

159. Preparation: The ingredients are initially introduced in the order given and mixed with water. The pH is adjusted to 5.5-6.5 using citric acid. The mixture is then homogenized for 10 minutes at 20° C. and the resulting liquid soap is poured into bottles.

### EXAMPLE 3

### Preparation of a Syndet Soap

160.

| | Component | % by weight |
|---|---|---|
| A | Sodium cocoylisethionate | 20 |
| | Sodium lauryl sulfoacetate | 16 |
| | Paraffin | 19 |
| | Wax, microcrystalline | 1 |
| B | Water | ad 100 |
| C | Antioxidant of the formula (14), (15), (17), (21), (22), | 0.002-0.05 |
| | (23), (25) or (28) | |
| | Maize starch | 8 |
| | Coconut fatty acid | 2 |
| | Lauryl diethanolamide | 2 |
| | Dextrin | 21 |
| | Lactic acid (88%) | 1 |

161. Preparation: the soap base (A) is thoroughly mixed, and water (B) is added at 20° C. The paste is homogenized using a rotor-stator, and then components (C) are added in the order given with vigorous mixing. The mixture is homogenized for a further 15 minutes and extruded in a bench extruder. The soap bars are produced by pressing (bench press).

### APPLICATION EXAMPLES

### EXAMPLE 4

### Test as Regards Discoloration in Soaps in an Oxidative Environment

162. Antioxidants tested: compounds of the formulae (11), (15), (16), (25), (32) and (33).

163. 500 ppm of the respective antioxidant to be tested are, together with 500 ppm of benzoyl peroxide and 0.2% of titanium dioxide, homogeneously distributed in a customary soap base (mixture of tallow fat, coconut and palm kernel soaps) by vigorous mixing and kneading in a bench mixer. The mixing process is repeated several times to ensure homogeneous distribution of the antioxidant in the soap.

164. The mixture is extruded using a bench extruder, producing test soap bars weighing approximately 1 g. These soaps are stored in a drying cabinet at 40° C. for two months, individual test soap bars being checked for their discoloration after each week. This is carried out by quantitative colorimetry, the reflectance spectroscopic method advantageously being used. This method is described in detail in the literature (see, for example, Colorimetry, Second Edition, International Commission on Illumination (CIE), CIE publication 15.2 (1986)). Comparison of the colorimetric results of the test preparations with those of the standard (freshly prepared sample having the same composition) is a measure of the colour stability of the antioxidant used.

165. It is found that a good colour stability is achieved with the antioxidants tested.

### EXAMPLE 5

### Test as Regards Solubility in Soaps

166. Antioxidants tested: compounds of the formulae (11), (15), (16), (21), (23), (24), (32) and (33).

167. To determine the solubility, 0.1% of each of the antioxidants is thoroughly mixed into a customary soap base (mixture of tallow fat, coconut and palm kernel soaps) at 40° C. The mixture is then cooled to room temperature and a light microscope is used to check whether the antioxidant is homogeneously distributed in the soap composition (absence of crystals, no mixed phases).

168. The results of these experiments show that the compounds tested have very good solubility and can be incorporated quickly and completely.

### EXAMPLE 6

### Test as Regards Stability to Hydrolysis

169. Antioxidants tested: compounds of the formulae (11), (14), (15), (16), (17), (21), (22), (24), (28), (32) and (33).

170. The stability of antioxidants in alkali can be tested quickly using a simple test. For this purpose, 0.05% of each of the antioxidants is mixed, in a bench mixer, into conventional soap flakes which customarily contain 10-15% of moisture and 0.02-0.1% of free alkali (as Na₂O) (overall values, % by weight). The mixing process should be repeated several times to ensure homogeneous distribution of the antioxidant in the soap. The samples are stored for 2 months in a drying cabinet at 40° C., and then the relative content of unchanged antioxidant is determined using HPLC analysis. The reference used (100% values) is the HPLC signals (heights or areas) of the respective antioxidants in freshly prepared soap formulations.

### RESULTS

171. The antioxidants tested have good stability in soap formulations. Their content in alkaline soaps, as determined by HPLC, is virtually unchanged after storage for 2 months in accordance with the above test.

172. The novel compositions (soaps) thus have good stability to colour changes and chemical decomposition.

### Antioxidants for the Stabilization of Formulations Comprising Surfactants

What is claimed is:

1. A cleaning composition comprising
(a₁) a phenolic antioxidant of the formula 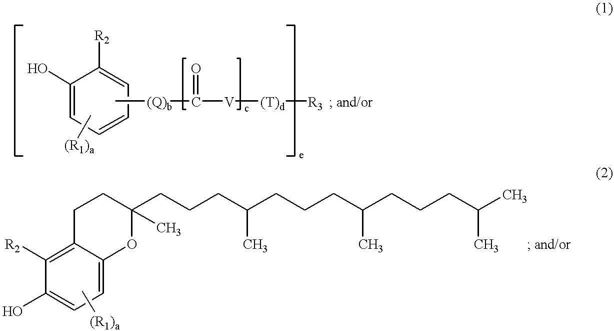
(a₂) an antioxidant of the formula 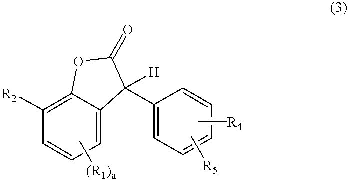
in which in the formulae (1), (2) and (3)
R₁ is hydrogen; C₁-C₂₂ alkyl; C₁-C₂₂ alkylthio; C₅-C₁₂ cycloalkyl; phenyl; or C₇-C₉ phenylalkyl;
R₂ is C₁-C₂₂ alkyl; C₅-C₁₂ cycloalkyl; phenyl; C₇-C₉ phenylalkyl; or —SO₃M;
Q is —CₘH₂ₘ—; 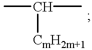
—CₘH₂ₘ—NH; a radical of the formula 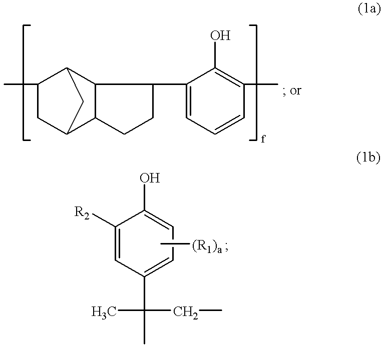
T is —CₙH₂ₙ—; —(CH₂)ₙ—O—CH₂—; 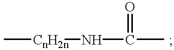
or a radical of the formula 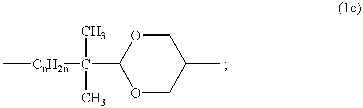
V is —O—; or —NH—;
a is 0; 1; or 2;
b, c and d independently of one another are 0; or 1;
e and f independently of one another are an integer from 1 to 3; and
m, n and p independently of one another are an integer from 1 to 3;
when e is 1,
R₃ is hydrogen; M; C₁-C₂₂ alkyl; C₅-C₁₂ cycloalkyl; C₁-C₂₂ alkylthio; C₂-C₂₂ alkenyl; C₁-C₁₈ phenylalkyl; a radical of the formula 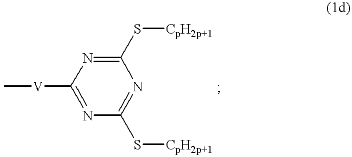 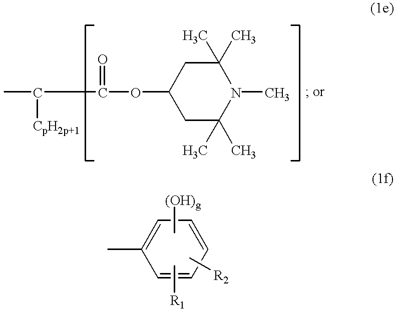
where, when R₃ is C₁-C₂₂ alkyl, b=0; or Q is a radical of the formula (1a) or (1b);
g is 0 or 1;
M is hydrogen; alkali metal; or ammonium;
when e is 2,
R₃ is a direct bond; —CH₂—; 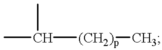
—O—; or —S—;
when e is 3,
R₃ is a radical of the formula 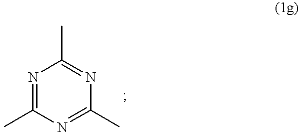 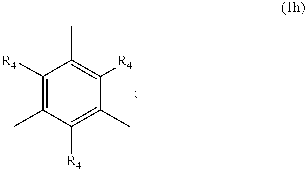 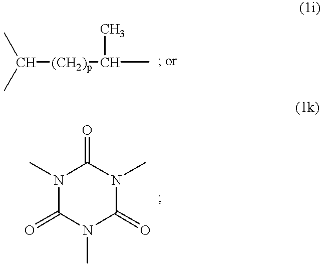
where, when R₃ is a radical of the formula (1k), c=1; and;
R₄ and R₅ independently of one another are hydrogen; or C₁-C₂₂alkyl; and
(b) a surfactant comprising a long alkyl or alkenyl chain.

2. A composition according to claim 1, wherein in formula (1)
Q is —CₘH₂ₘ—,
in which
m is as defined in claim (1).

3. A composition according to claim 1, wherein
Q is a methylene or ethylene radical.

4. A composition according to claim 1, wherein
V is —O—.

5. A composition according to claim 1, wherein
R₁ and R₂ independently of one another are C₁-C₂₂ alkyl.

6. A composition according to claim 5, wherein
R₁ and R₂ independently of one another are C₁-C₅ alkyl.

7. A composition according to claim 1, wherein
a is 1.

8. A composition according to claim 1, wherein component (a₁) is a compound of the formula 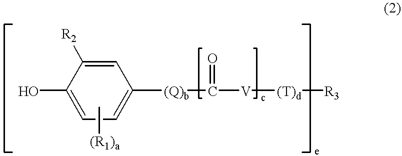
in which
R₁ and R₂ independently of one another are C₁-C₅ alkyl,
a is 1 or 2; and
R₃, Q, V, T, b, c, d and e are as defined in claim 1.

9. A composition according to claim 8, wherein
e is 1.

10. A composition according to claim 9, wherein compounds of the formula 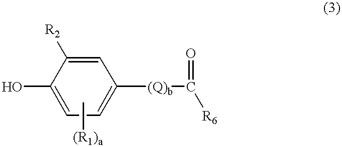
are used, in which
R₆ is —O—M; or 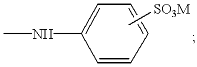
M is hydrogen; ammonium; or alkali metal; and
R₁, R₂, Q, a and b, are as defined in claim 8.

11. A composition according to claim 1, wherein component (a₁) is a compound of the formula 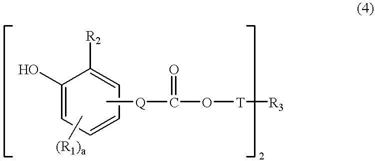
in which
R₁ and R₂ independently of one another are C₁-C₅ alkyl;
Q is —CₘH₂ₘ—; or —CₘH₂ₘ-NH—;
R₃ is a direct bond; —O—; —S—; or a radical of the formula (1f);
a is 1 or 2;
m is 1 to 5; and
T is as defined in claim 1.

12. A composition according to claim 11, wherein
Q is ethylene;
R₃ is a direct bond; and
R₁, R₂, T and a are as defined in claim 11.

13. A composition according to claim 11, wherein
T is —O—CH₂—.

14. A composition according to claim 1, wherein compounds of the formula 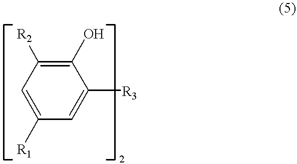
are used, in which
R₁ and R₂ are C₁-C₅-alkyl; and
R₃ is —CH₂— or

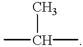

15. A composition according to claim 1, wherein component (a₁) is a compound of the formula 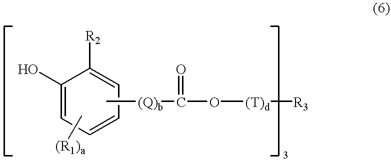
in which
Q is —CₘH₂ₘ—;
T is —CₙH₂ₙ—;
R₁ and R₂ independently of one another are C₁-C₅ alkyl;
R₃ is the radical of the formula (1g); (1h); (1i); or (1k);
m and n independently of one another are from 1 to 3;
a is 1 or 2; and
b and d independently of one another are 0 or 1.

16. A composition according to claim 15, wherein compounds of the formula 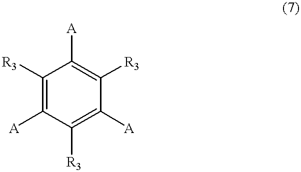
are used, in which
A is a radical of the formula 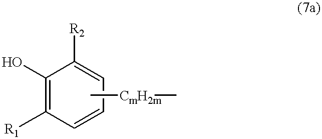
R₁, R₂ and R₃ independently of one another are C₁-C₅ alkyl; and
m is 1 to 3.

17. A composition according to claim 15, wherein compounds of the formula 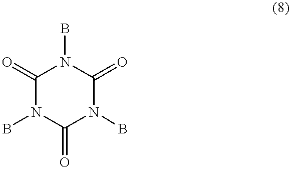
are used, in which
B is a radical of the formula 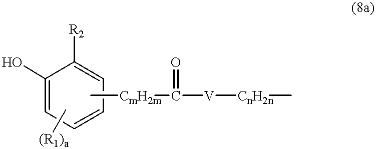
R₁ and R₂ independently of one another are C₁-C₅ alkyl;
V is —O—; or —NH—;
a is 1; or 2;
m is 1 to 3; and
n is 0 to 3.

18. A cleaning composition according to claim 1, wherein the antioxidants corresponding to the component (a₁) and (a₂) are used as an individual compound or a mixture of several individual compounds.

19. A cleaning composition according to claim 1, wherein the component (a₁) or (a₂) or the total of components (a₁) and (a₂) is present in a concentration of from 50 to 1000 ppm.

20. A cleaning composition according claim 1, wherein component (b) is an anionic, nonionic or zwitterionic and amphoteric synthetic, detersive substance.

21. A cleaning composition according to claim 20, wherein the anionic detersive substances are alkali metal salts, ammonium salts or amine salts of C₈-C₂₀ fatty acids.

22. A cleaning composition according to claim 1, in which component (c) is a light protection agent of the sterically hindered amine type.

23. A cleaning composition according to claim 22, in which component (c) is a 2,2,6,6-tetraalkylpiperidine derivative which contains at least one group of the formula 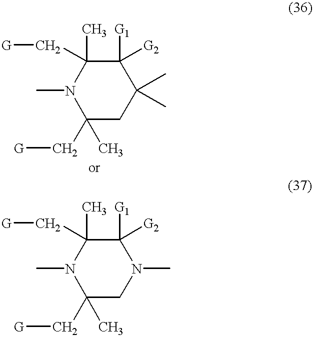
in which
G is hydrogen or methyl.

24. A cleaning composition according to of claim 1 in the form of a solid, gel, syndet or liquid formulation.
